(19) United States
(12) Patent Application Publication
(10) Pub. No.: US 2010/0086992 A1
(43) Pub. Date: Apr. 8, 2010

(54) BIOSENSOR, BIOSENSOR CHIP AND METHOD FOR PRODUCING THE BIOSENSOR CHIP FOR SENSING A TARGET MOLECULE

(75) Inventors: Michael Himmelhaus, Chuo-ku (JP); Spencer Spratt, Chuo-ku (JP)

Correspondence Address: SUGHRUE MION, PLLC, 2100 PENNSYLVANIA AVENUE, N.W., SUITE 800, WASHINGTON, DC, 20037, US

(73) Assignee: Fujirebio Inc., Chuo-ku, Tokyo (JP)

(21) Appl. No.: 12/518,156

(22) PCT Filed: Dec. 21, 2007
(86) PCT No.: PCT/JP2007/075345

§ 371 Date: Nov. 18, 2009

Related U.S. Application Data
(60) Provisional application No. 60/871,765, filed on Dec. 22, 2006.

(51) Int. Cl. C12M 1/34; B05D 3/00

(52) U.S. Cl. 435/287.1; 427/2.11

(57) ABSTRACT

A biosensor chip for sensing a target molecule, includes: a substrate having a surface with a sensing area; and adhesive material for immobilizing a mollicute having a cell membrane on the sensing area. The chip may includes cell-resistant material for preventing the mollicute from being immobilized on those parts of the surface of the substrate that do not belong to the sensing area. Further, the adhesive material may comprise a first adhesive material for immobilizing a body of the mollicute on the sensing area and a second adhesive material for immobilizing a tip of the mollicute on the surface of the substrate.

### TECHNICAL FIELD

The present invention relates to a technology for a biosensor, a biosensor chip and a method for producing the biosensor chip for sensing a target molecule.

The entire contents of the prior U.S. Provisional Applications No. 60/796,162 filed on May 1, 2006, and No. 60/871,765 filed on Dec. 22, 2006, are incorporated herein by reference.

### BACKGROUND ART

Whole cells as bio-interfaces of biosensors have been suggested by many authors. For a recent review, see T.-H. Park and M. L. Shuler, Biotechnol. Progr., Vol. 19, pp. 243-253, 2003. Besides discussing the potential of cells for biosensing, the article also provides insight into the techniques used for micropatterning and surface-grafting of cells. One interesting approach in this context is the so-called Cell Culture Analogue (CCA), which is an artificial device that mimics cell function. CCA are mainly targeting pharmaceutical research, but could have—after proper adaptation—also potential for biosensing.

One crucial aspect of the utilization of cells for biosensing is their controlled adhesion onto micro- or nanopatterns. Such art is important not only for fundamental studies in cell biology, such as cell adhesion, cell growth, metabolism, and apoptosis, but further aiming at applications in tissue engineering, neuroscience, and the development of man-machine interfaces. Nanopatterns are particularly useful when molecular definition of cell binding points and multivalent interactions are crucial [S. Svedhem et al., ChemBioChem, Vol. 4, pp. 339-343, 2003; Ch. Selhuber et al., Nano Letters, Vol. 6, pp. 267-270, 2006]. However, the cells used for adhesion in these studies have mesoscopic size with average diameters of typical several tens to few hundreds of micron. No attempts have been reported in the literature, where the cells grafted on the nanopatterns have sub-micron dimension by themselves.

Besides whole cells, also cell membranes can be used to introduce activity and high selectivity into the biological recognition process of a biosensing system. Also here, a variety of methods have been applied, ranging from the surface adsorption of natural cells [M. Tanaka et al., Phys. Chem. Chem. Phys., Vol. 3, pp. 4091-4095, 2001] to spreading of artificially formed lipid bilayers [E. Sackmann, Science, Vol. 271, pp. 43-48, 1996] as membrane substitutes. While usage of the latter is much simpler and easier to control, they exhibit disadvantages in terms of lower mechanical and chemical stability and insufficient fluidity of the bilayer, in particular after application of freezing and thawing cycles. For more detailed information, see the following reviews and articles: E. Sackmann, Science, Vol. 271, pp. 43-48, 1996; J. T. Groves et al., Science 275, pp. 651-653, 1997; M. Tanaka & E. Sackmann, Nature, Vol. 437, pp. 656-663, 2005; 0. Worsfold et al., Langmuir, Vol. 22, pp. 7078-7083, 2006; M. Tanaka et al., J. Am. Chem. Soc., Vol. 126, pp. 3257-3260, 2004.

Mollicutes have been studied extensively, in particular in biomedical sciences due to their parasitic nature. Many of them cause diseases in plants, animals, and humans, such as mycoplasma pneumonia or mycoplasma arthritidis. However, besides being directly malignant for the host organism, mollicutes also provide an elegant way of infiltration for other pathogens, in particular viruses, which then may cause severe infection of the host. For an extensive review on these topics, inventors of the present invention refer to the book “Molecular Biology and Pathogenicity of Mycoplasmas”, edited by Shmuel Razin and Richard Herrmann, Kluwer Academic/Plenum Press, New York, 2003 [ISBN 0-306-47287-2] and the reviews by S. Razin et al. on the topic [S. Razin et al., Microbiol. Mol. Bio. Rev., Vol. 62, pp. 1094-1156, 1998; S. Razin, Physiol. Rev., Vol. 83, 417-432, 2003].

One other field of science, where mollicutes have been investigated vastly, are studies on the properties and function of the cell membrane. This is founded by the fact that mollicutes show a permanent lack of the outer cell wall. They basically comprise a biologically active, fully functional plasma membrane directly accessible from the outside. Much of today's knowledge about fluidity and function of membrane bilayers was gained by studies on mollicutes. While no attempts have been made so far to apply mollicute membranes directly to biosensing, the fruitfulness of this field of research highlights the feasibility of the approach suggested in the present document. For further information, the inventors refer to S. Rottem and I. Kahane (eds.), “Mycoplasma Cell Membranes”, Subcell. Biochem., Vol. 20, pp. 1-314, Plenum Press, New York 1993 and, e.g., M. E. Tourtellotte et al., Proc. Natl. Acad. Sci., Vol. 66, pp. 909-916, 1970.

The only known application of mollicutes to the vastly evolving field of bio-nanotechnology published so far is not related to biosensing, but to molecular transport and development of molecular machines. Hiratsuka and coworkers report on the use of a fast gliding mycoplasma (mycoplasma mobile) as a transporter for biological agents in microtracks [Y. Hiratsuka et al., Biochem. Biophys. Res. Comm., Vol. 331, pp. 318-324, 2005].

The development of nano-biosensors is driven by a plurality of reasons of academic as well as industrial relevance. Obviously, smaller sensors require less analyte, promise improved signal-to-noise ratio, lower production costs, and fit overall better in our increasingly miniaturized world. Further, biomedical and pharmaceutical research demand for high throughput screening of analytes, for example for screening of the entire human genome on a single biochip. Such giant data processing strongly demands for small feature size to become manageable. The most exciting aspect, however, is related to the fact that nanoscale sensors reach into the dimensions of our nanostructured biological world, thereby opening the opportunity to detect biological processes locally right at their venue. Arrays of nano-biosensors could trace mass transport and changes in concentration of biological analytes locally, e.g. across single cells. Such highly resolved sensing may open an entirely new world for scientists and researchers working in many different fields, ranging from basic science to clinical research, drug development, tissue engineering, the development of artificial organs and implants, and man-machine devices.

A typical concept for the construction of a biosensor is to combine a fully functional biochemical structure that bears specifically binding ligates targeting the desired molecules, to a physical device that acts as transducer for the conversion of biological events into machine-readable data. For a nano-biosensor this means, that such hybridization of biological and physical compounds must occur on a sub-micron level. While due to the rapid progress in nanotechnology and information processing, a variety of physical devices are available that provide mainly electronic, optical, or optoelectronic transducer mechanisms with sub-micron scale resolution, it turned out to be rather difficult to functionalize such devices with fully operable biochemical structures. This is the more surprising since in particular biological matter is considered for its huge hierarchy in structures, reaching from molecular dimensions up to macroscopic scale. The scale on which nano-biosensing operates therefore should be easily accomplishable. However, biochemical events, such as specific recognition processes, are highly dynamic in nature and are more likely a subtle balance of many competitive processes rather than a single reaction. Therefore, the challenge is to provide a fully functional biochemical unit composed of all relevant building blocks within sub-micron dimension.

In practice, the challenge of fabricating biochemical interfaces that can selectively bind a target molecule with high specificity can be narrowed to two basic requirements: presence of specific ligates and potential of suppressing non-specific binding of a plurality of other molecules, which might be present in the same sample. Secondary demands, which are more important for commercialization, are related to lifetime and storage issues as well as a high reliability and reproducibility of the fabrication process.

The first basic requirement, the need for a specific ligate, is relatively easy to fulfill since nature herself is the inventor of the lock-key principle involved in specific recognition processes. Therefore, the entire problem can be reduced to the isolation and handling of the wanted ligates after they have been produced in a suited host organism. In the past decades, a variety of techniques have been developed, e.g. for the growth and harvesting of monoclonal antibodies, which in a second step can be physically or chemically attached to the physical sensor. The challenge, however, is to guarantee high specificity by suppressing non-specific binding events. The latter is of utmost importance, since the physical transducer mechanism in general cannot distinguish between molecules adhering to the sensor due to specific or non-specific interactions, because the nature of the forces involved (electrostatic, van-der-Waals, hydrophobic) is basically the same for both types of interactions. Therefore, specificity can only be introduced by a highly selectively acting biochemical structure as interface between biological and physical compound of the sensor.

A standard method to suppress non-specific interactions is based on exposing the biochemical structure bearing the ligates to other, adhesive proteins, such as bovine serum albumine (BSA), in order to block nonspecific adsorption sites. However, the efficiency of this method depends on both the substrate used and the biological system under study, and exchange processes may occur between dissolved and surface-bound species (Vroman effect). Therefore, recently a variety of attempts were made to integrate the specific ligates into a matrix material, which resists nonspecific protein adsorption. Candidate matrix materials with excellent protein repulsive properties are for example thin films of polyethylene glycol) (PEG) [E. W. Merrill: Poly(Ethylene Oxide) and Blood Contact in J. M. Harris, Ed.; Plenum Press: New York, 1992, pp 199-220; C.-G. Gölander, J. N. Herron, K. Lim, P. Claesson, P. Stenius, J. D. Andrade: Properties of Immobilized PEG Films and the Interaction with Proteins: Experiments and Modeling in J. M. Harris, Ed.; Plenum Press: New York, 1992; pp 221-245] and oligo(ethylene glycol) (OEG) [K. L. Prime and G. M. Whitesides, Science 1991, vol. 252, pp. 1164-1167; K. L. Prime and G. M. Whitesides, J. Am. Chem. Soc. 1993, vol. 115, pp. 10714-10721].

However, besides some success on laboratory scale with simple biological model fluids, biochemical interfaces using such films could not show sufficient stability and reproducibility, which would make them suitable for industrial scale applications. A particular problem of ethylene glycol derivates is, e.g., their instability with respect to oxidation.

Another problem with such artificial biochemical interfaces, which has been not mentioned so far, is the activity of the ligates used for specific binding. While the production and harvesting of such ligates is no problem, their implementation into the biochemical interface by adsorption or chemical binding can cause their degeneration and thus loss of their activity. All these problems, i.e. insufficient specificity and activity of the biochemical interface of a biosensor, may be overcome, when a natural host matrix for embedding the ligates is chosen. Cell membranes, for example, contain a plurality of different specifically acting ligates, which remain altogether active and highly specific despite of the presence of a highly complex biological environment [E. Sackmann, Science 1996, vol. 271, pp. 43-48].

Therefore, it seems to be a reasonable attempt to utilize cell membranes as the biochemical interface in biosensing. Natural cell membranes harvested from human or animal cells however are difficult to utilize in particular in view of industrial scale production. First of all, the membranes are very complex and contain a variety of proteins and receptors that might cause side effects, such as unwanted specific interactions, in biosensing. Further, natural variation in their composition complicates reliability and process control.

Therefore, many attempts have been made to fabricate artificial surface-supported membranes, which can be used as model cell surfaces and enriched with those molecules necessary for the respective study [M. Tanaka and E. Sackmann, Nature 2005, vol. 437, pp. 656-663].

### DISCLOSURE OF INVENTION

While current technology allows the fabrication of artificial surface-supported membranes on large scale, i.e. on homogeneous surfaces, patterned membranes are so far restricted to the micron size regime. Nanopatterning of artificial membranes has not been achieved so far and seems to be difficult due to insufficient stability. Further, the functionality of artificial membranes on such small scale is questionable, since fluidity of the cell membrane is of utmost importance for its proper function. However, fluidity has been found to be difficult to achieve with artificial membranes [O. Worsfold, N. H. Voelcker, T. Nishiya, Langmuir 2006, vol. 22, pp. 7078-7083]. Restricting the total dimension of the membrane to sub-micron dimensions will further complicate this problem.

From the above it becomes clear that the fabrication of a fully operable biochemical interface comprising high activity and specificity of the integrated ligates as required for biosensing remains a major challenge. This is particularly true with respect to industrial scale production of biosensors, which in addition to the above requirements further demands for important practical properties, such as good storage behavior, long lifetime, easy production and so on.

The present invention has been achieved in order to solve the problems which may occur in the related arts mentioned above.

A biosensor chip for sensing a target molecule according to one aspect of the present invention, includes: a substrate having a surface with a sensing area; and adhesive material for immobilizing a mollicute having a cell membrane on the sensing area.

A biosensor for sensing a target molecule according to another aspect of the present invention, includes: the biosensor chip; a transducer for detecting changes in mass or refractive index on the sensing area; and a flow cell providing the biosensor chip with analyte.

A method for producing a biosensor chip for sensing a target molecule according to another aspect of the present invention, includes: preparing a substrate having a surface with a sensing area; and disposing adhesive material on the sensing area for immobilizing a mollicute having a cell membrane on the sensing area.

### BRIEF DESCRIPTION OF THE DRAWINGS

FIG. 1 is a schematic view that depicts a first basic scheme for immobilizing mollicutes on nanopatterns according to an embodiment of the present invention, wherein FIG. 1(a) shows nanopatterns which are formed by adhesive material and cell-resistant material on a substrate, and FIG. 1(b) shows the nanopatterns with the mollicutes which are immobilized by the adhesive material on the substrate.

FIG. 2 is a schematic view that depicts a second basic scheme for immobilizing mollicutes on nanopatterns according to another embodiment of the present invention, wherein FIG. 2(a) shows nanopatterns which are formed by two different adhesive materials and the cell-resistant material on the substrate, and FIG. 2(b) shows the nanopatterns with mollicutes whose tips are immobilized by one of the adhesive materials and whose bodies are immobilized by the other of the adhesive materials on the substrate.

FIG. 3 is a schematic view that depicts a first scheme for embedding ligates into a cell membrane of the mollicutes, wherein FIG. 3(a) shows ligates which are covalently attached to lipid molecules before the lipid molecules assemble into the cell membrane, and FIG. 3(b) shows ligates attached to lipid molecules which are assembled into the cell membrane.

FIG. 4 is a schematic view that depicts a second scheme for embedding ligates in the mollicutes by means of genetic engineering, wherein FIG. 4(a) shows a mollicute with a natural DNA, FIG. 4(b) shows a mollicute where a foreign sequence has been inserted into the DNA, and FIG. 4(c) shows a mollicute with a modified DNA after expressions of the wanted ligates in its cell membrane.

FIG. 5 is a schematic view that depicts a scheme (I) for manufacturing a biosensor chip utilizing the mollicute as biochemical interface.

FIG. 6 is a schematic view that depicts a scheme (II) for manufacturing the biosensor chip having two sensing surfaces on the substrate.

FIG. 7 is a schematic view that depicts a scheme (III) for manufacturing the biosensor chip in which the mollicute is immobilized in an oriented fashion.

FIG. 8 is a schematic view that depicts utilization of the biosensor chip.

FIG. 9 is a schematic view that depicts an example for a biosensor for sensing a target molecule by utilizing the biosensor chip.

FIG. 10 is a schematic view that depicts potential reflective properties of cavity surfaces, wherein FIG. 10(a) shows the properties in the case of a non-metallic cavity, and FIG. 10(b) shows the properties in the case of a metal-coated cavity.

FIG. 11 is a schematic view that depicts a simplifying estimation for the wavelength of cavity modes of micro-cavities, wherein FIG. 11(a) shows the estimation for metal-coated cavities, and FIG. 11(b) shows the estimation for non-metallic cavities.

FIG. 12 shows Scanning Electron Microscopy (SEM) micrographs of Acholeplasma Laidlawii (APL) cells immobilized on a silicon substrate after different periods of growth; FIG. 12(a) shows APL cells after two days of growth in the culture medium, while FIG. 12(b) shows APL cells after six days of growth; FIG. 12(c) is a close-up of the top right corner of FIG. 12(b).

FIG. 13 demonstrates the feasibility of integrating lipid-labeled probe molecules into APL membranes; FIG. 13(a) is a confocal fluorescence image of a cluster of APL cells stained with a lipid-labeled fluorophore, while FIG. 13(b) is a confocal transmission image of the same cluster acquired simultaneously with the fluorescence image.

FIG. 14 shows the results of the experiments on biospecific interactions using lipid-biotin labeled APL cells in suspension; FIG. 14(a) gives the result of the experiment using fluorescent-labeled Streptavidin as a specifically binding target molecule (platereader set to Rhodamine B detection), while FIG. 14(b) shows the results using fluorescent bovine serum albumine (BSA) as non-specifically interacting target molecule (platereader set to Alexa Fluor 488 detection); Fluorescence intensities shown are normalized to the intensity measured under the same conditions for PBS buffer alone.

FIG. 15 gives the results of the experiments on biospecific interactions using surface-immobilized lipid-biotin labeled APL cells (platereader set to Rhodamine B detection); Fluorescence intensities shown are normalized to the intensity measured under the same conditions for PBS buffer alone.

FIG. 16 shows SEM micrographs of the surfaces evaluated in FIG. 15. Micrographs of FIG. 16 and results bars of FIG. 15 with same label correspond to each other.

FIG. 17: Results of experiments on biospecific interactions using surface-immobilized lipid-biotin labeled APL cells; (I) cells exposed to Rhodamine B-labeled Streptavidin, plate reader set for detection of Rhodamine B; (II) cells exposed to Alexa-Fluor 488-labeled BSA, plate reader set for detection of Alexa-Fluor 488; (III) cells of (I) after additional exposure to Alexa-Fluor 488-labeled BSA, plate reader set for detection of Alexa-Fluor 488; (IV) cells of (II) after additional exposure to Rhodamine B-labeled Streptavidin, plate reader set for detection of Rhodamine B; Fluorescence intensities shown are normalized to the intensity measured under the same conditions for PBS buffer alone.

FIG. 18: SEM micrographs showing close-ups of surface-adsorbed APL cells used for the experiment shown in FIG. 15.

FIG. 19: SEM micrographs of nanopatterned APL cells,

wherein FIG. 19(a) shows APL cells patterned on Si patches with a nominal diameter of 3 μm, FIG. 19(b) shows a control pattern of 3 μm Si patches exposed to the culture medium only, FIGS. 19(c) and (d) display APL cells patterned on Si patches of about 1 μm nominal diameter, and FIG. 19(e) shows an APL cell patterned on a ˜500 nm Si patch.

FIG. 20: APL cells grown from a probe of cells that had been frozen at −30 centigrades for three days.

### BEST MODE FOR CARRYING OUT THE INVENTION

Exemplary embodiments relating to the present invention will be explained in detail below with reference to the accompanying drawings.

Basic Concepts

First, basic concepts of the embodiments are explained below. Based on the above observations, the inventors have perceived a surprisingly easy and straightforward solution to all these problems, in particular with respect to the fabrication of biochemical interfaces of sub-micron dimension. In addition, the solution provides the potential of incorporating also the ligate production, such as monoclonal antibodies, into the fabrication of these biochemical interfaces.

Suggestion by the inventors is to utilize mollicutes, such as mycoplasma or acholeplasma, as the biochemical interface. Mollicutes are the smallest self-replicating cells known with sizes down to few hundreds of nanometers. Due to their small size, their genome length belongs to the shortest ever known. Therefore, mollicutes have limited biosynthetic capabilities and many of them cannot produce all the proteins required for survival and self-replication by themselves. Instead, they spend their life as parasites docking to matured plant, animal or human cells by membrane fusion (S. Rottem, Physiological Reviews, Vol. 83, pp. 417-432, 2003). Once docked to a higher developed cell, these mollicutes supply themselves with all essential proteins and biomolecules required from the host cell. Probably for this reason, the cell membrane fusion, mollicutes do not comprise an outer cell wall like plant or animal cells (S. Razin et al., Microbiology and Molecular Biology Reviews, Vol. 62, pp. 1094-1156, 1998). They are simply confined by the double layer of the lipid membrane.

Altogether, these unique properties of mollicutes have made them very attractive for a variety of studies. Genome research is interested in the smallest genome required for self-replication. In this context mollicutes are called the “quantum bits of life”. Due to the lack of the outer cell wall, they are very popular with cell membrane studies.

Almost all of our knowledge about the fluidity and the physicochemical properties of cell membranes originate from studies on mollicutes (L. Rilfors et al., “Regulation and Physiochemical Properties of the Polar Lipids in Acholeplasma Laidlawii” in Subcellular Biochemistry, Vol. 20, pp. 109-166, ed. S. Rottem and I. Kahane, Plenum Press, New York, 1993; R. N. McElhaney, Biochimica et Biophysica Acta, Vol. 779, pp. 1-42, 1984). Finally, their parasite life is also of interest for biomedical research. Due to the lacking outer cell wall mollicutes are very easy to penetrate by viruses. Thus, when docked to a matured plant, animal, or human cell, they provide easy access ports for virus infections, such as HIV, SARS, etc. that target the host cells.

Therefore, mollicutes are thought to play a key role in the infectious pathways of viral diseases (S. Razin et al., Microbiology and Molecular Biology Reviews, Vol. 62, pp. 1094-1156, 1998).

The present idea of using mollicutes as nanoscale biochemical interfaces for biosensing relies on several of these unique properties. Since some mollicutes dock to matured cells, their membranes comprise a high number of cell adhesion molecules and receptors (S. Rottem, Physiological Reviews, Vol. 83, pp. 417-432, 2003). Thus, they may be very easily surface-adsorbed onto nano-patches by patterning a flat surface, i.e. the sensing surface of the physical signal transducer, with proper cell adhesion molecules, such as integrins or other suitable ligates. To facilitate intrusion into host cells, some mollicutes further exhibit a so-called “tip”, which contains special ligates not present in their body (cf., eg. J. Hegermann et al., Naturwissenschaften, Vol. 89, pp. 453-458, 2002 and references therein; S. Rottem, Physiological Reviews, Vol. 83, pp. 417-432, 2003). Therefore, it seems to be feasible to achieve surface immobilization in an oriented and controlled fashion by taking advantage of the presence of different types of ligates in the tip and body of the mollicute.

Basic Schemes for the Surface Adsorption of Mollicutes

The mollicutes can be immobilized on a surface of a substrate by at least two different basic schemes explained below.

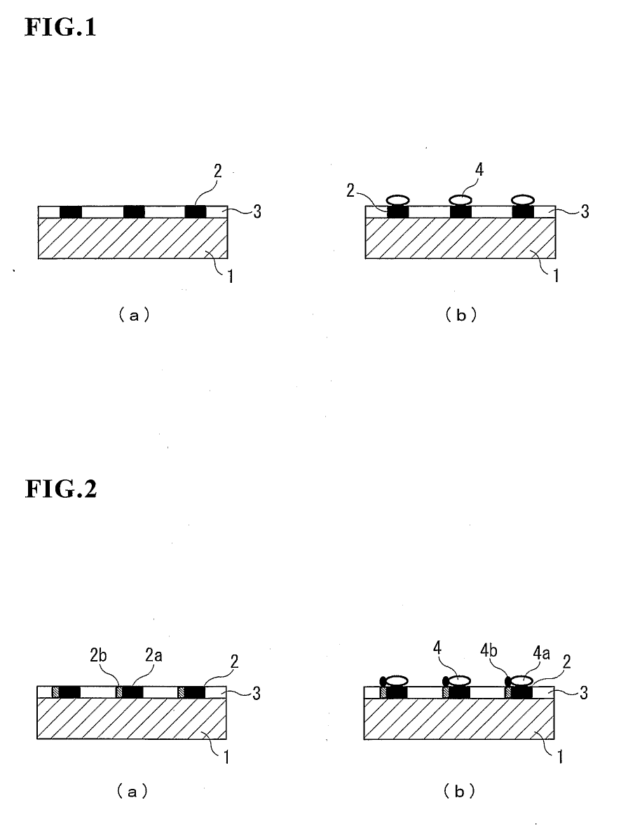

FIG. 1 displays the first basic scheme for the immobilization of mollicutes 4 which are with random orientation immobilized onto a nanopattern formed on a surface of a substrate 1. As shown in FIG. 1(a), the nanopattern, which is formed by standard nanopatterning techniques as known by those skilled in the art on a suitable substrate 1, consists of a cell adhesion material 2 and a cell-resistant material 3. The cell adhesion material 2 is a nano-size patch for promoting mollicute adhesion, e.g. due to the presence of cell adhesion molecules. The cell-resistant material 3 is for preventing mollicutes 4 being adhered on the surface of the substrate 1. Thus, as shown in FIG. 1(b), mollicutes 4 can be attached on the cell adhesion material 2 but not on the cell-resistant material 3. Accordingly, positioning of mollicutes 4 on the substrate 1 can be accomplished in this first basic scheme.

FIG. 2 displays the second basic scheme which extends the concept of the first scheme. The second scheme utilizes cell adhesion material 2 containing two different adhesion materials 2a and 2b, each of which utilizing different cell adhesion molecules. The first adhesion material 2a targets the body 4a of the mollicutes 4, while the second adhesion material 2b is specific only to the tip 4b of the mollicutes 4 by means of suitable cell adhesion molecules. Accordingly, oriented immobilization of the mollicutes 4 can be accomplished in this second basic scheme.

Next, the details of materials which can be used as the substrate 1, the cell adhesive material 2, the cell resistant material 3, or the mollicutes 4 are explained.

As the substrate 1, any materials can be used that can be nano-patterned. Preferably, materials that are well-established in micro- and nanofabrication will be used, such as silicon wafers, glasses, quartz, indium tin oxide, germanium, gallium arsenide and related composite semiconductors, and polymers suitable for nanofabrication, such as polymethylmethacrylate, polydimethylsiloxane, polystyrene, polyimide, and others. Further, metals and their thin films deposited on a semiconducting or insulating substrate (for example those materials described above) can be used, such as the coinage metals (gold, silver, copper, platinum), aluminium, cobalt, nickel, iron, titanium, and their oxides. For deposition of the adhesive materials 2a and 2b and of the cell resistant material 3 onto the substrate 1 in a controlled fashion, the substrate 1 can for example be nanopatterned in a sense that any suitable composite of the materials mentioned above can be fabricated and used as substrate. Then, each component of the substrate 1 exposed to the surface is selective for only one of the cell adhesive material 2 and the cell resistant material 3, respectively. Accordingly, the pattern formed by the cell adhesive material 2 and the cell resistant material 3 on the substrate 1 after their deposition mimics the pattern of the composite substrate 1.

In general, other fabrication schemes for micro/nanopatterning known to those skilled in the art may be used to fabricate patterns suitable for random or oriented mollicute patterning, for example also those schemes utilizing direct writing lithography (e.g., e-beam or dip-pen lithography) and/or applying steps of destructive patterning processes, such as reactive ion etching.

As the cell adhesive materials 2a and 2b, any kind of biomolecule specifically binding to mollicutes can be used, such as proteins, peptides, antibodies, nucleotides, and receptors. For example, short peptide sequences, such as linear and cyclic RGD and PHSRN are known to bind specifically to cell adhesion molecules of the cell surface, such as integrins.

Besides integrins, a variety of other cell-adhesive molecules, such as immunoglobulins (IgCAM), selectins, cadherins, heparin sulphate proteoglycans, ADAMs (cell surface proteins containing A Disintegrin and A Metalloprotease), and protein tyrosine phosphatases, may be present in a cell membrane. They can be specifically targeted by proper choice of a specific linker molecule, such as those described above. For example, an antibody can be designed such that it targets specifically at a single type of cell-adhesive molecule. Antibodies targeting specifically at a single type of integrin are already commercially available. Further, extracellular matrix proteins, e.g. those that are produced by colony-forming mollicutes, may be used to provide surface functionalization suitable for mollicute patterning. Recently, some mollicute genomes have been fully sequenced (e.g. Acholeplasma Laidlawii PG-8A: G. Y. Kovaleva et al., Kharkevich Institute, Moscow, Russia, source: NCBI reference NC_{—}010163) so that also methods of bio-formatics and/or genetic engineering may be used to identify and/or (over-)express suitable surface linker molecules either in the entire membrane or the tip or body of the mollicute only. Independent of the method chosen, selective targeting of tip and body of a mollicute may then be achieved by the distribution, i.e. the relative concentrations, of the cell adhesion molecules across the mollicute. The tip of mollicutes is known to contain particular cell adhesion molecules, which are not present or present only in marginal amounts in its body.

For the cell resistant material 3, any kind of the known materials can be used, such as polyethylene glycol, BSA, dextran, phosphorylcholine [A. L. Lewis, Colloids and Surfaces B, Vol. 18, pp. 261-275, 2000], N-isopropylacrylamide [T. Bohanon et al., J. Biomater. Sci. Polym. Ed., Vol. 8, pp. 19-39, 1996], and their derivatives.

For the mollicutes 4, one of the smallest mollicutes known is Acholeplasma Laidlawii (APL), which is found in animals, and present for example in cattle, bovine milk and related products, and is classified as biosafety level 1 by the American Type Culture Collection. Thus, despite of its parasitic nature, it is not pathogenic and no special care is required for its utilization. Therefore, in the following, the inventors will discuss their approach by using APL as an example. However, one should keep in mind that it might turn out that other, currently less studied mollicutes will serve this purpose even better in the future. The diameter of a surface adsorbed APL ranges from about 350 to 1200 nm and thus may fit the requirement of a submicron biochemical interface. Further, as outlined by Wieslander and coworkers, the size of APL may be controlled in this regime by addition of lipid molecules (Edman et al., The Journal of Biological Chemistry, Vol. 278, pp. 8420-8428, 2003). APL is very robust, i.e. it can be frozen and thawed without any loss of fluidity and thus activity of its cell membrane. Further, as a natural object surviving in harsh environments, APL (and mollicutes in general) provide antioxidants in their cell membrane to prevent their membranes from denaturation. Oxidation is in fact one of the major problems in the utilization of artificial cell membranes.

Schemes for Embedding Ligates into the Membrane of Mollicutes

Specific ligates can be embedded into the cell membrane of the mollicutes by at least two different schemes explained below.

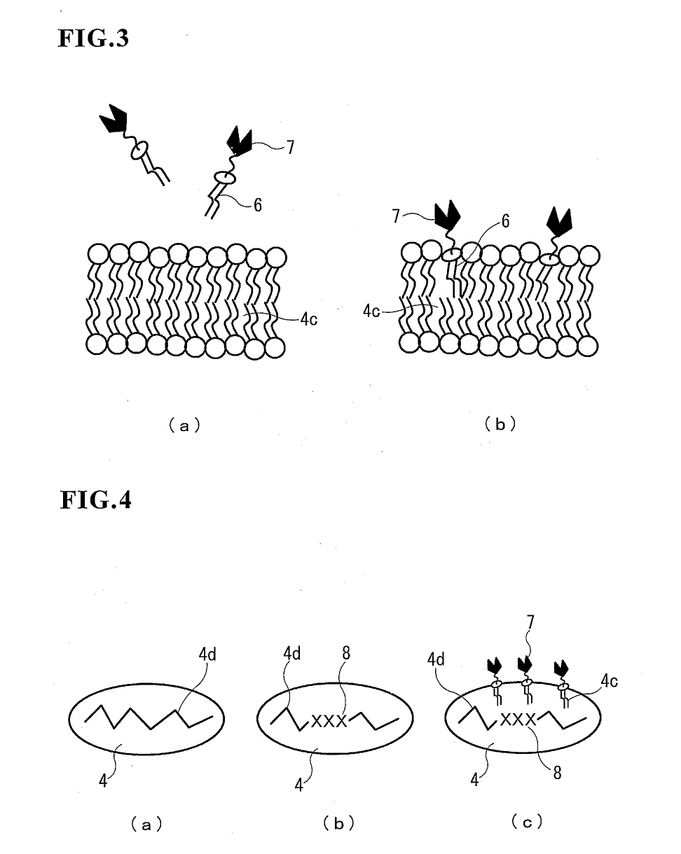

FIG. 3 shows the first scheme for embedding the ligates. In the first scheme for embedding, the specific ligates 7 are simply embedded into the cell membrane 4c of APL (or other mollicutes) 4 by attaching them to a lipid molecule 6. In aqueous solution, the lipid molecule 6 then penetrates into the membrane 4c, leaving the water-soluble ligate 7 on the outside. Examples of such art are given e.g. in Worsfold et al. [O. Worsfold, C. Toma, T. Nishiya, Biosens. and Bioelectron. 19 (2004) 1505-1511]. As shown in FIG. 3(a), a ligate 7 is covalently attached to a lipid molecule 6. Then, as shown in FIG. 3(b), the lipid molecule 6 having the ligate 7 assembles into the cell membrane 4c of mollicutes 4 in an aqueous environment.

FIG. 4 shows the second scheme for embedding the ligates. In the second scheme for embedding, the specific ligates 7 are introduced into the cell membrane 4c of APL (or other mollicutes) 4 by genetic engineering. The DNA sequence of several mollicutes, such as mycoplasma pneumonia, is already known. Simple cutting procedures for insertion and replacement of DNA sequences have been developed (cf. e.g. “Molecular Biology and Pathogenicity of Mycoplasmas”, edited by Shmuel Razin and Richard Herrmann, Kluwer Academic/Plenum Press, New York, 2003 [ISBN 0-306-47287-2] and references therein). Therefore, similar to the production of antibody-like molecules, proteins or hybrids by genetic engineering (cf. eg. G. Kohler & C. Milstein, Nature, Vol. 256, pp. 495, 1975; Pluckthun, EP0324162; Inaba et al., US2005/0064557 A1; P. J. Hudson and C. Souriau, Nature Medicine, Vol. 9, pp. 129-134, 2003), direct expression of the ligates wanted for biosensing seems to be feasible. For example, as shown in FIG. 4(a), a mollicute 4 having the natural DNA 4d is prepared. Then, as shown in FIG. 4(b), the natural DNA 4d is cut and a foreign sequence 8 is inserted into the mollicute 4. Under suitable conditions, as shown in FIG. 4(c), the mollicute 4 starts to express the wanted ligates 7 in its cell membrane 4c. Further, exogenous DNA, such as plasmids or bacteriophages, can be used for the same purpose. In this scheme, the ligates can be embedded into the cell membrane of the mollicute by any genetic engineering methods including: by modifying a inherent DNA sequence of the mollicute by at least one sequence required for the expression of the biomolecule in a cell membrane or by transforming a plasmid or bacteriophage into the mollicute so that the mollicute can express the biomolecule in a cell membrane of the mollicute. The word “modifying” in this context includes replacing the inherent DNA sequence by the sequence required for the expression of the biomolecules and inserting the sequence required for the expression of the biomolecules into the cell membrane.

Schemes for Producing the Biosensor Chip

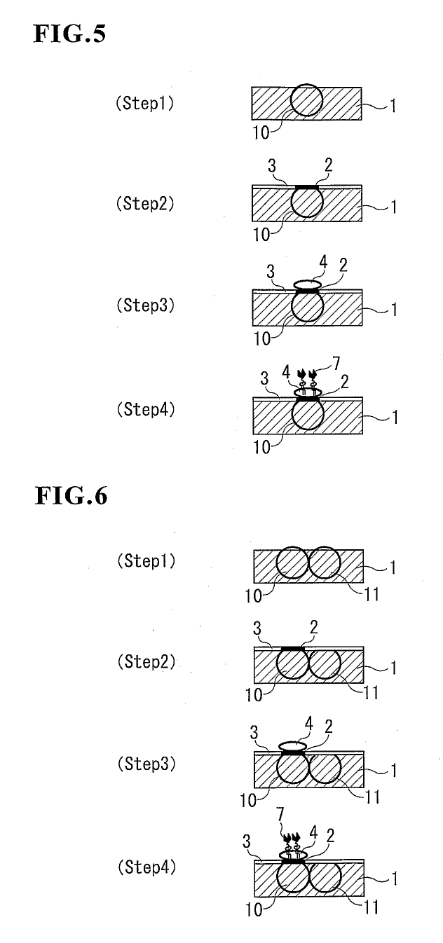

Simple three schemes for producing the biosensor chip are displayed in FIGS. 5-7.

In the first scheme shown in FIG. 5, the cavity 10 (a particle to define the cavity 10) is embedded in the substrate (a host material) 1 such that only a sub-micron patch of its surface is exposed outside of the surface of the substrate 1 (step 1). The surface of the substrate 1 is then coated with the cell-resistant material 3, while the exposed area of the cavity 10 is coated with the cell adhesion material 2 (step 2). After immobilization of the mollicute 4 onto the cell adhesion material 2 (step 3), suitable ligates 7 are either embedded into the cell membrane from the outside or produced by the mollicute due to prior genetic engineering of its DNA (step 4). Note that the coating by the cell-resistant material 3 must properly function only through the immobilization process of the mollicute 4. For later sensing of specific interactions, non-specific adsorption onto the coating by the cell-resistant material 3 can be tolerated, since the optical transducer is not sensitive in these areas.

In the second scheme shown in FIG. 6, this principle of the above scheme is extended to the presence of a second optical cavity 11, which is embedded in the substrate 1 (step 1). The surface of the substrate 1 including the exposed area of the second cavity 11 is then coated with the cell-resistant material 3, while the exposed area of the cavity 10 is coated with the cell adhesion material 2 (step 2). After immobilization of the mollicute 4 onto the cell adhesion material 2 (step 3), suitable ligates 7 are embedded into the cell membrane (step 4). The second optical cavity 11 is not biofunctionalized, but serves as a reference sensor accounting for changes in temperature or refractive index of the medium, etc. Also, the reference may measure the amount of non-specific adsorption onto the coating by the cell-resistant material 3.

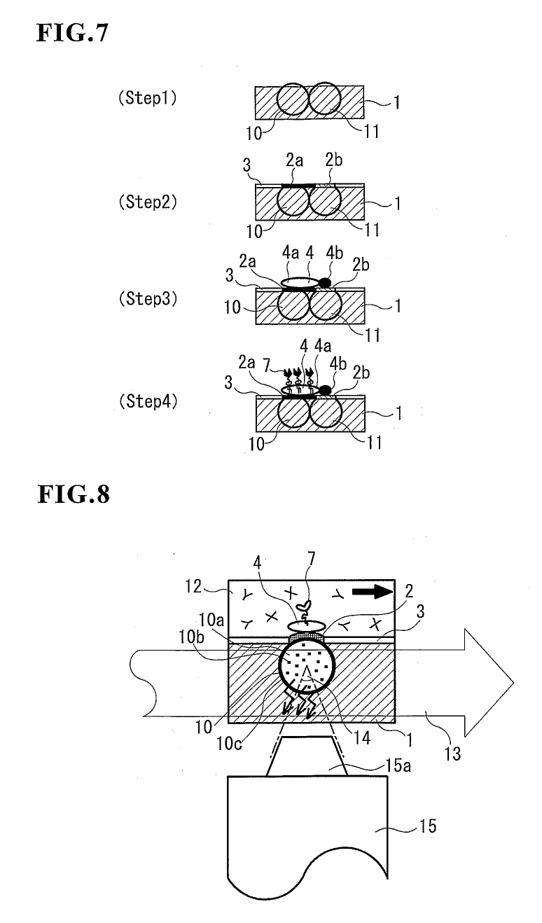

In the third scheme shown in FIG. 7, finally, an application of oriented mycoplasma adsorption is shown. After the first cavity 10 and the second optical cavity 11 are embedded in the substrate) (step 1), similar to the scheme shown in FIG. 2, the first adhesion material 2a and the second adhesion material 2b are introduced into the nanopatterns (step 2). The second adhesion material 2b is specifically targeting the tip 4b of the mollicute 4, while the first adhesion material 2a is specifically targeting only to the body 4a of the mollicute 4. Accordingly, the mollicutes 4 adhere in an oriented fashion (step 3). The ligates 7 are then embedded into the cell membrane (step 4). In this third scheme, it is exemplified that the tip 4b attaches to the reference cavity 11, e.g. to correct the biosensor signal originating from the optical cavity 10 for any changes caused by unwanted activity of the mollicute 4.

The sensor can be frozen for storage. After thawing, the APL may either still be alive, e.g. to produce fresh ligates, or alternatively, undergo cell death to avoid self-replication or other unwanted activity. In any case, the fluidity of the cell membrane, and thus its unique activity and specificity with respect to antibody/antigen or ligate/ligand binding will be maintained.

Advantages of Using Mollicutes

In the following, a summary of the advantages of using mollicutes in biosensing as biochemical interfaces on submicron scale is given:

| Problem typical for | |
|---|---|
| biosensing | Solution by use of mollicutes |
| Non-specific interactions | Fluid membranes highly resistant |
| | to non-specific binding |
| Insufficient ligate activity | Fluid membrane provides optimum |
| | ligate acitivity |
| Maintenance of fluidity in | Mollicutes are surface attached as |
| surface-immobilized | entire cells; nevertheless, their cell |
| membranes | membrane stays active and fluid. |
| | Mollicutes are trained for survival |
| | in harsh environments. |
| Oxidation of the cell | Mollicute membranes contain |
| membrane | antioxidants for survival in harsh |
| | environments. |
| Loss of membrane fluidity | Mollicute membranes contain |
| after freezing | sugars and related reagents to |
| | maintain fluidity of the cell |
| | membrane also after freezing. |
| Nanopatterning of cell | Mollicutes are already of |
| membranes or biochemical | submicron dimension. Using |
| interfaces for biosensing | standard techniques of |
| in general | nanofabrication allows their |
| | immobilization onto nanopatterned |
| | surfaces, thereby achieving the |
| | wanted biochemical interface in a |
| | single step. |
| Production of | Gene engineering of mollicutes |
| ligates/antibodies and | may be used for ligate production |
| insertion at the wanted | right on site, even after mollicute |
| position on a surface | immobilization on nanopatterns. |
| | Accordingly, the technique might |
| | play a key role also for the |
| | fabrication of complex ligate arrays |
| | consisting of a plurality of different |
| | ligates (bio array fabrication). |

Biosensor Utilizing the Biosensor Chip

Next, a biosensor for sensing a target molecule utilizing the biosensor chip mentioned above is explained. Here, a basic physical transducer mechanism is based on optical sensing by means of an optical microcavity [F. Vollmer et al., Appl. Phys. Lett. 2002, vol. 80, pp. 4057-4059; S. Arnold et al., Optics Letters 2003, vol. 28, pp. 272-274]. Further, details of an optical element utilized in the biosensor is according to the provisional application No. 60/796,162.

As illustrated in FIG. 8, the cavity 10 includes a non-metallic core 10a containing a fluorescent material 10b and a metallic coating 10c enclosing the non-metallic core 10a. The substrate 1 is transparent for the excitation and emission wavelengths of the fluorescent material 10b. The exposed part of the cavity 10 is coated with the cell-resistant material 3 (a protein-resistant matrix) and the cell adhesion material 2. The mollicute 4 is immobilized by the cell adhesion material 2 and the ligates 7 is embedded into the cell membrane of the mollicute 4. The surface of the substrate 1 is mounted into a liquid cell 12 in such a way that the exposed and biofunctionalized surface of the cavity 10 (namely, the ligates 7) comes into contact with the analyte contained in the liquid cell 12. The fluorescent material 10b is optically pumped by a light beam 13, which propagates through the substrate 1, thereby also traversing the cavity 10. The light emitted from the fluorescent material 10b and transmitting through the metallic coating 10c of the cavity 10 due to the finite Q-factor (see following definition) of the cavity 10 is collected within a certain solid angle 14 by an optical fibre 15. The solid angle 14 is given by the numerical aperture of the fibre 15 and its distance from the centre of the cavity 10. For small cavities 10 with diameters below 1 μm, the tip 15a of the optical fibre 15 can fabricated such that it allows sub-wavelength resolution (Optical near field tip) to provide proper discrimination of the signal from noise. Typically, such a sharpened tip is controlled by means of a scanning optical near-field microscope (SNOM). Alternatively, the light emitted by the cavity can be collected by means of a far-field set-up, such as a microscope equipped with a suitable objective. In any case, the light can be guided to the detection system for analysis.

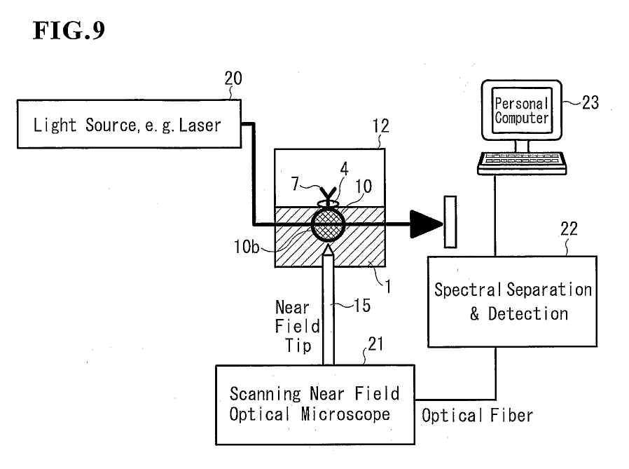

Particles or particle systems embedded in or supported by a solid substrate 1 can be operated as biosensors by means of the following setup shown in FIG. 9. The substrate 1 is mounted into a liquid cell 12 to allow the exposure of the ligates 7 to a medium containing potential specific binding partners. The fluorescent material 10b is excited by means of a light beam generated by a laser or another suitable light source 20, while the emission from the cavity 10 is collected by means of a suitable optical system, e.g. an optical fibre 15. The fibre then guides the light to an optical analysis system (an optical microscope 21, an spectral separation and detection system 22, and a personal computer 23) which records the intensity of the detected light as a function of wavelength and time.

In a preferred embodiment, the light source used for excitation of the fluorescent material 10b is an ultrashort pulse laser, while the detection unit 22 is able to discriminate ultrashort signals from noise. The latter can be implemented by means of a gated CCD camera or a photomultier connected to a fast processing electronics, such as a boxcar integrator. Ultrashort pulse lasers with sufficiently short pulses in the nano-, pico-, and femtosecond regime are commercially available.

Definition of Terms

The definition of terms which are used in the above explanation is described below. Definition of other terms is according to the provisional application No. 60/796,162.

Ligate: A ligate is a (bio-)molecule, such as a receptor, antibody, or protein, capable of specifically binding a target molecule, also called “ligand”; as such, the ligate may be used in a biosensor to capture the wanted ligand so that it may be detected by the biosensor.

Probe molecule: Used as synonym for ligate.

Biofunctional interface: A biofunctional interface is a surface or interface between a physical transducer used for the detection of a biological event and a biological environment that is capable of binding specifically a wanted target molecule (ligand), while other unwanted, nonspecific interactions are suppressed.

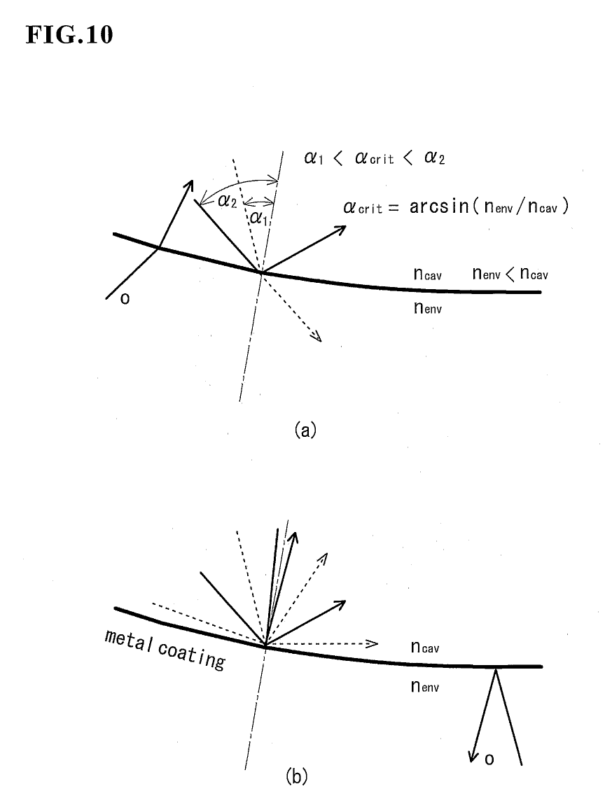

Cavity (Optical cavity): An optical cavity is a closed volume confined by a closed boundary area (the “surface” of the cavity), which is highly reflective to light in the ultraviolet (UV), visible (vis) or infrared (IR) region of the electromagnetic spectrum. Besides its wavelength dependence, the reflectance of this boundary area may also be dependent on the incidence angle of the light impinging on the boundary area with respect to the local surface normal (cf. FIG. 10). The inner volume of the optical cavity may consist of vacuum, air, or any material that shows high transmission in the UV, vis, or IR. In particular, transmission should be high at least for a part of those regions of the electromagnetic spectrum, for which the surface of the cavity shows high reflectance.

An optical cavity is characterized by two parameters: First, its volume V, and second, its quality factor Q. In the following, the term “optical cavity” refers to those optical cavities with a quality factor Q>1.

Volume of an optical cavity: The volume of an optical cavity is defined as its inner geometrical volume, which is confined by the surface of the cavity, i.e. the highly reflective boundary area.

Quality factor: The quality factor (or Q-factor) of an optical cavity is a measure of its potential to trap photons inside of the cavity. It is defined as

$$Q = \frac{stored energy}{loss per roundtrip} = \frac{{ω}_{m}}{Δ {ω}_{m}} = \frac{{λ}_{m}}{Δ {λ}_{m}} , \qquad ( 1 )$$

where ωₘ and λₘ are frequency and wavelength of cavity mode m, respectively, and Δωₙ, and Δλₘ are the corresponding linewidths. The latter two equations connect the Q-factor with position and linewidth of the optical modes inside of the cavity. Obviously, the storage potential of a cavity depends on the reflectance of its surface. Accordingly, the Q-factor is wavelength dependent.

Optical cavity mode: An optical cavity mode or just “cavity mode” is a wave solution of the electromagnetic field equations (Maxwell equations) for a given cavity. These modes are discrete and can be numbered with an integer m due to the restrictive boundary conditions at the cavity surface. Accordingly, the electromagnetic spectrum in presence of the cavity can be divided into allowed and forbidden zones. The complete solution of the Maxwell equations consists of internal and external electromagnetic fields inside and outside of the cavity, respectively. In the following, the term “cavity mode” refers to the inner electromagnetic fields inside the cavity unless otherwise stated. The wave solutions depend on the shape and volume of the cavity as well as on the reflectance of the boundary area, i.e. the cavity surface. Therefore, the solutions depend on the Q-factor of the cavity and its wavelength dependence.

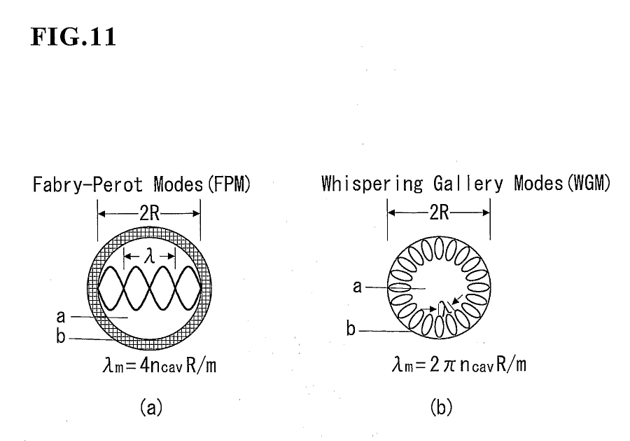

For spherical cavities, there exist two main types of solutions, for which the wavelength dependence can be easily estimated. For simplicity, these estimates will be used in the discussion below. FIG. 11 illustrates the difference between the two. We assume that in both cases a standing wave has formed. In FIG. 11(a) the standing wave formed in radial direction, while in FIG. 11(b) it formed along the circumference of the inner boundary between sphere and environment (in the case of a sphere coated with a metallic shell, the standing wave forms along the inner shell boundary). These standing waves can be viewed at as superpositions of counterpropagating traveling modes in either radial or azimuthal direction, respectively. In the following, we will call the modes in radial direction “Fabry-Perot Modes” (FPM) due to analogy with Fabry-Perot interferometers. The modes forming along the circumference of the spheres are called “Whispering Gallery Modes” (WGM) in analogy to an acoustic phenomenon discovered by Lord Rayleigh. For a simple mathematical description of the wavelength dependence of these modes, we use the standing wave boundary conditions in the following (for illustration, cf. FIG. 11):

λₘ=4Rn_{cav}/m, m=1, 2, 3, (2)

for FPM, which states that the electric field at the inner particle surface has to vanish for all times, as is the case e.g. for a cavity with a metallic coating. For WGM, the standing wave condition yields

λₘ=2πRn_{cav}/m, (3)

for WGM, which basically states that the wave has to return in phase after a full roundtrip. In both formulas, “m” is an integer and is also used for numbering of the modes, R is the sphere radius, and n_{cav} the refractive index inside of the cavity.

Mode volume of a cavity mode: The mode volume of a cavity mode is defined as that geometrical volume, where the field intensity of the mode is not vanishing. Since in general the fields are decaying exponentially, a certain cut-off value defining “zero intensity” has to be set in practise. For example, the cut-off can be fixed to 0.1% of the maximum field intensity.

Biosensors Utilizing the Biosensor Chip in General

Besides the biosensor described above, any kind of biosensor that is capable of sensing specific binding to the biosensor chip is applicable. While the sensor described above is capable of sensing with nanoscale lateral resolution, i.e. the sensing area may have submicron dimension and may contain a single mollicute, such high resolution is not required. In case of low lateral resolution, i.e. a sensing area with dimension in the micrometer or even millimeter regime, the biosensor chip can be made on the respective scale by placing many submicron sensing areas as described above, each of which containing a single or several mollicutes, in a proper distance of each other on the large scale surface. Then, the sensor measures the specific binding to these multitude of individual submicron sensing areas on average. The only difference to the biosensor as described above is that particular care has to be taken to prevent non-specific binding on this large scale surface, since it would affect the average signal measured by the low resolution biosensor across the large scale surface.

Examples of suitable sensors with low lateral resolution (i.e. resolution in the micron or millimeter regime) are: evanescent field sensors, such as fiber sensors and surface plasmon sensors (SPR; e.g. the biacore system, see, http://www.biacore.com); reflectometric sensors, such as reflectometers and ellipsometers; sensors based on holography and interference (e.g. surface holograms as developed by Smart Holograms, see, http://www.smartholograms.com); mass sensing sensors, such as quartz microbalances (e.g. the Q-sense system, see, http://www.q-sense.com) and related acoustic wave sensors.

Examples for suitable sensors with high lateral resolution, i.e. capable of sensing a single submicron sensing area containing as few as a single mollicutes, are:

Scanning probe techniques, such as atomic force microscopy (AFM), near field optical microscopy (SNOM), electron and X-ray microscopies, and localized surface plasmon sensors.

### EXAMPLES

### Example 1

### Growth of Acholeplasma Laidlawii (APL)

As an example of a mollicute with sub-micron dimension that can be used for biosensing, Acholeplasma Laidlawii (APL) was chosen. Particular advantages of using APL are related to the unique properties of its cell membrane in terms of fluidity and accessibility (R. N. McElhaney, Biochimica et Biophysica Acta, Vol. 779, pp. 1-42, 1984; L. Rilfors et al., “Regulation and Physiochemical Properties of the Polar Lipids in Acholeplasma Laidlawii” in Subcellular Biochemistry, Vol. 20, pp. 109-166, ed. S. Rottem and I. Kahane, Plenum Press, New York, 1993; R. N. McElhaney, Critical Reviews in Microbiology, Vol. 17, pp. 32, 1989), the ease of culturing (E. B. Stephens et al., The Yale Journal of Biology and Medicine, Vol. 56, pp. 729-735, 1983), and its potential for genetic engineering (T. K. Jarhede and Ake Wieslander, Methods in Molecular Biology, Vol. 104, pp. 247-258, in Mycoplasma Protocols, ed. R. J. Miles and R. A. J. Nicholas, Humana Press, Totowa, N.J., USA). In this example, a procedure for culturing and growing APL is described along with a method for fixing surface-adsorbed cells to study their shape and appearance by means of scanning electron microscopy (SEM).

### Experimental:

### Acholeplasma Laidlawii Medium Preparation

17.5 g of Heart Infusion Broth (HIB (BD 238400)) were dissolved in 700 ml of MilliQ (MQ) H₂O. 38 ml aliquots of HIB solution were autoclaved and stored at 4° C. until required. Mycoplasma medium (MycoM (American Type Culture Collection (ATCC) Medium 243)) was made up by adding 5.5 ml of Yeast Extract solution (Gibco 18180-059) and 11 ml of Horse Serum (heat inactivated) (Gibco 26050-070) to the HIB aliquot.

### Growing Acholeplasma Laidlawii

An Acholeplasma Laidlawii strain A (APL (ATCC 14089)) glycerol stock kept at −80° C. was stabbed with a 1 ml Gilson pipette with an extended filter tip and used to inoculate a solution of MycoM in a 200 ml conical bottle with a screw cap over a Bunsen flame. The APL culture was left in the bottle with a slightly loosened lid, shaking at 80 rpm in a water bath (IWAKI, SHK-101B)) set at 37° C. until required.

Fixing Acholeplasma Laidlawii on a Surface for Scanning Electron Microscopy

APL culture was aliquoted into 1 ml eppendorf's and centrifuged (KUBOTA 3740) at 10,000 g for 20 minutes. The MycoM supernatant was discarded and the APL in each tube were resuspended in 1 ml of Phosphate buffered saline solution (PBS). The tubes were centrifuged again as before. The PBS wash was discarded and the APL resuspended in the desired volume of PBS. The Absorbance of the APL solution was then measured using a Spectrophotometer (Beckman) set at 260 nm obtaining a reading of approximately 2.0. APL suspension was then added to Silicon chips in the wells of a 6-well plate (353046, Falcon) and left for 2 hours. Chips were then immersed straight into PBS 4% Glutaraldehyde for 1 hour. After fixation the chips were washed with MQ H₂O from a wash bottle and dried with N₂ from a pressurized cylinder. Surfaces were then coated with 10 nm of gold by using an evaporator at about 4×10⁻⁵ hPa nitrogen pressure to achieve an isotropic coating of cells and surface with gold. The chips were then looked at using SEM (Hitachi S-4200).

### Results:

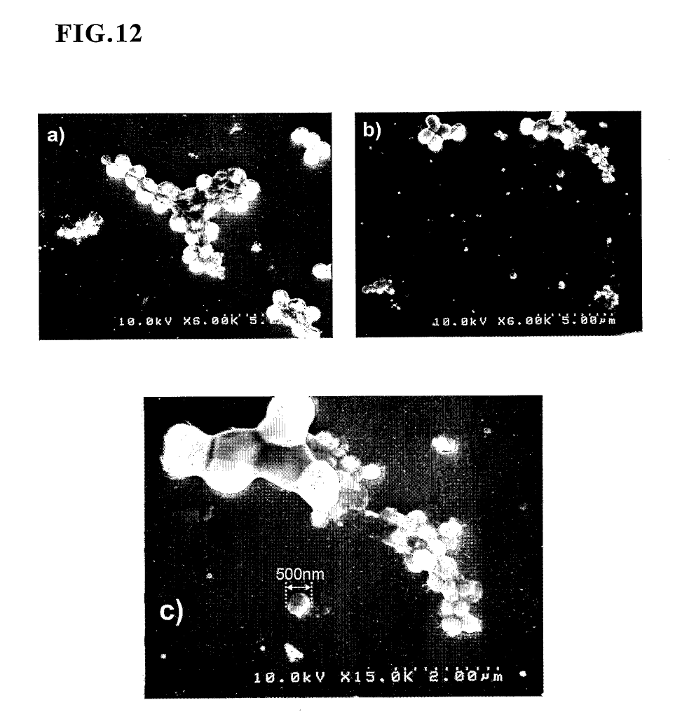

FIG. 12 shows cells after two (FIG. 12(a)) and six (FIGS. 12b and 12c) days of growth. The cells seem to form grape-like colonies. Two main sizes can be found in the culture. The larger size amounts to about 1.2 μm diameter for a single cell, the smaller one to 300-500 nm diameter. This can be seen nicely in the close-up of FIG. 12(b) shown in FIG. 12(c). The two sizes can be used, e.g., to prepare biofunctional interfaces of different extension from a single cell.

### Example 2

### Accessibility of the Cell Membrane of Acholeplasma Laidlawii

Mollicutes exhibit a permanent lack of their outer cell wall, so that the lipid membrane is directly accessible and can be easily used for the integration of specific probe molecules, e.g. by linking them to a lipid or fatty acid molecule that self-assembles into the membrane as illustrated in FIG. 3.

Experimental: As an example, APL was grown and after 2 days was washed and resuspended in PBS as described in Example 1. Then, a fluorophore-tagged fatty acid, 4,4-difluoro-5-(2-thienyl)-4-bora-3a, 4a-diaza-s-indacene-3-dodecanoic acid (BODIPY 558/568 C₁₂; D3835, Molecular Probes) in DMSO, was added to the APL suspension at 0.01 mM and left gently shaking for 1 hour. The tubes were then centrifuged as before, the supernatant discarded and the cells were resuspended in 1 ml PBS. This last step was repeated to assure that all dye molecules not attached to the cell membranes are removed from the suspension. The cell suspension was then added to the well of a 6-well plate and made up to 3 ml with PBS and observed by laser scanning confocal microscopy and light transmission microscopy using an Olympus Fluoview 1000 with a LUMPlan FI 60×/0.90 W objective.

### Results:

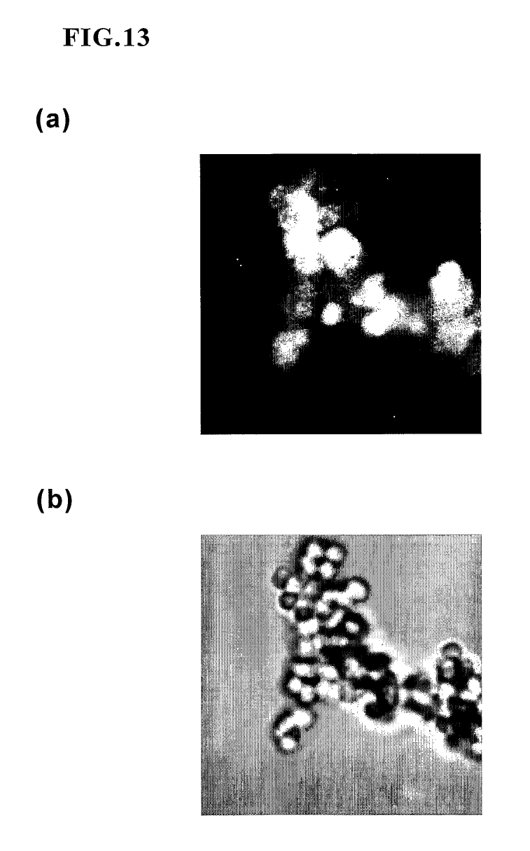

FIG. 13 shows simultaneously acquired confocal fluorescence and transmission images of a cluster of APL cells after staining with the fluorophore-tagged fatty acid. Obviously, the dye molecules have stained the cells completely, i.e., the entire outer cell surface. Since we are using a fatty acid molecule that is known not to react otherwise with cells than integrating into the membrane, the feasibility of using APL cell membranes as biofunctional interfaces via membrane-integrated probe molecules is demonstrated with this example.

### Example 3

### Specific Interactions Using Lipid-Anchored Probe Molecules

After demonstrating the feasibility of accessing the lipid membrane of APL, the next step is to show that the membrane can be used for specific recognition of a target molecule using a probe molecule artificially integrated into the cell membrane. As a specifically interacting pair, biotin/Streptavidin was chosen. The biotin was covalently attached to a lipid, while the Streptavidin was fluorophore-tagged to allow the tracking of successful binding events.

### Experimental:

An APL suspension in PBS was prepared as described in Example 2. Lipid-Biotin (LiB) (16:0 Biotinyl Cap PE (870277, Avanti Polar Lipids, Inc.)) at 2 mg/ml in ethanol or Lipid (16:0 PE (850705P, Avanti Polar Lipids, Inc.) at 1 mg/ml or Biotin (Sigma) or nothing was sonicated for 10 minutes and briefly vortexed before adding to APL solution in the desired volume in eppendorf's in triplicate at 0.04 mM. The solution was then left gently shaking for 1 hour.

After 1 hour the Lipid-Biotin labeled APL (APL-LiB) solution and controls were centrifuged as before, the solution was discarded and the APL-LiB and controls were resuspended in 1 ml of PBS. The APL-LiB and control suspensions were centrifuged as before and the cells resuspended in the desired volume of PBS and pooled.

Streptavidin-Rhodamine B (SRB) (S871, Molecular Probes) was added to 200 μl the APL-LiB and controls at 0.0001 mM in eppendorf's and left gently shaking for 1 hour. Next the tubes were centrifuged as described previously. 100 μl of the supernatant was added to wells of a 96-well plate (353072, Falcon). The rest of the supernatant was discarded and the APL were washed by resuspension in 1 ml PBS. This cell suspension was then centrifuged as before. 100 μl of this wash was then added to wells of a 96-well plate before discarding the remainder. APL were resuspended in the original volume of PBS and 1041 added to wells of the 96-well plate. The relative fluorescence in each well was then measured using a plate reader (SAFIRE, TECAN).

As a test for non-specific sensing the same protocol was followed as described in the above section except Alexa Fluor 488 labeled BSA (BSA488) (A13100, Molecular Probes) was used instead of SRB.

### Results:

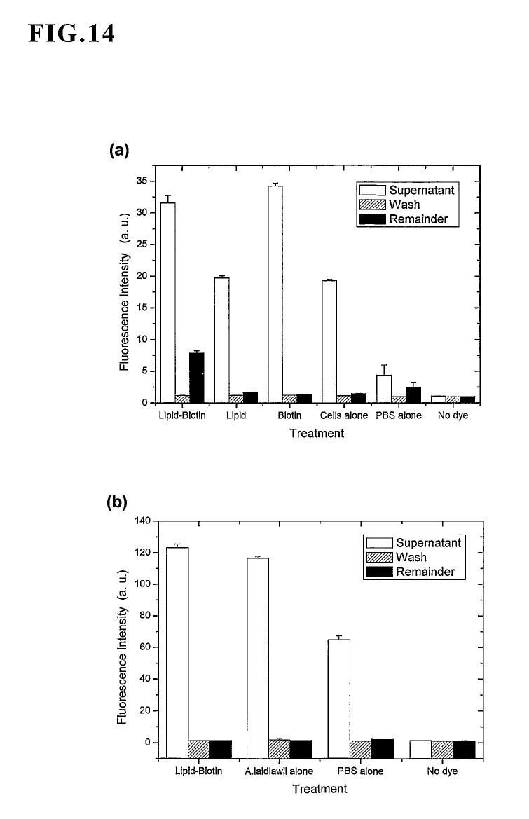

FIG. 14 shows the results of the platereader measurements. The signals of supernatant and wash can be viewed at as estimates for maximum and minimum signals, i.e. they roughly indicate the measurement range. Accordingly, the signal of the remainder is mostly in-between these two results. As can be seen from FIG. 14(a), the remainder signal has basically the same intensity as the wash, i.e. minimum intensity, except for those APL cells labeled with the lipid-bound biotin. Neither pure lipid nor pure biotin or any other of the controls was able to achieve significant fluorescence intensity. This indicates that the Streptavidin binds specifically to the biotin inserted into the cell membrane of APL. That the interaction is in fact specific can be seen from FIG. 14(b) where lipid-biotin labeled cells were exposed to a fluorescent BSA molecule instead of Streptavidin. However, an increase in fluorescence intensity above the background as indicated by the wash cannot be observed. This proves that the cells bound Streptavidin to the lipid-anchored biotin inserted into their lipid membrane by specific interaction. Thus, the feasibility of specific recognition by means of a artificially introduced probe molecule into the lipid membrane of APL has been successfully demonstrated.

### Example 4

### Specific Interactions Using Surface-Immobilized Acholeplasma Laidlawii and Lipid-Anchored Probe Molecules

Example 3 showed that APL can be used as substrates for specific interactions using membrane-integrated probe molecules. For the development of an on-chip biosensor it is important to demonstrate that also surface-immobilized mollicutes can be used for the same purpose. Therefore, the experiment presented in example 3 is repeated with surface-adsorbed APL cells in the following.

### Experimental:

APL were labeled with LiB as described in Example 3. The same controls were also included. During the labeling process 1041 of the supernatant, the wash and the final APL suspension were kept and added to wells of a Poly-D-Lysine 96-well plate (354461, Becton Dickinson) and left for 2 hours. The solutions were then removed from each well and 100 μl of PBS was added. The PBS wash was then removed and 200 μl of PBS 1% BSA (A-7030, Sigma) was added to each well and left for 2 hours gently shaking. The BSA blocking buffer was then removed and 100 μl of PBS was added. The PBS wash was then removed and 100 μl of PBS with 0.0002 mM of SRB was added to each well and left for 1 hour gently shaking. The solutions were then removed and added to another 96-well plate for future reference. 100 μl of PBS wash was then added to each well. The plate was gently tapped and this wash was then removed and added to another 96-well plate for future reference. 100 μl of PBS was then added to the wells and the plate was read using the plate reader as before.

After reading the plate the APL on the surface were fixed by removing the solution in each well and adding 100 μl of PBS 4% Glutaraldehyde and left for 1 hour. After 1 hour the fix was removed and the wells washed several times with MQ H₂O and then left to air dry. The bottoms of the wells were cut out with a mini circular saw equipped with a diamond blade and 10 nm of gold was deposited (see Example 1) onto them. The surfaces were then looked at using SEM.

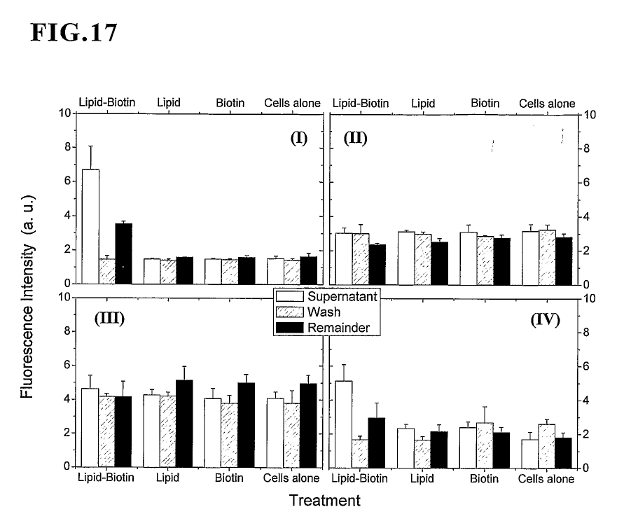

The same protocol was followed for the test on non-specific interactions as shown in FIG. 17, however, with the following inclusions: This time the APL labeling, including controls, was carried out in two lots of triplicates. The triplicate supernatants, washes and final APL suspensions were added to two halves of a Poly-D-Lysine 96-well plate. In addition to the 100 μl of PBS with 0.0002 mM of SRB added to first half of the plate, 100 μl of PBS with 0.0002 mM of BSA 488 was added to the second half of the plate. After a first plate reading, the respective fluorophores were again added to the plate but this time in reverse: i.e, 100 μl of PBS with 0.0002 mM of BSA 488 was added to the first half of the plate and 100 μl of PBS with 0.0002 mM of SRB was added to the second half the plate. A second plate reading was carried out.

### Results:

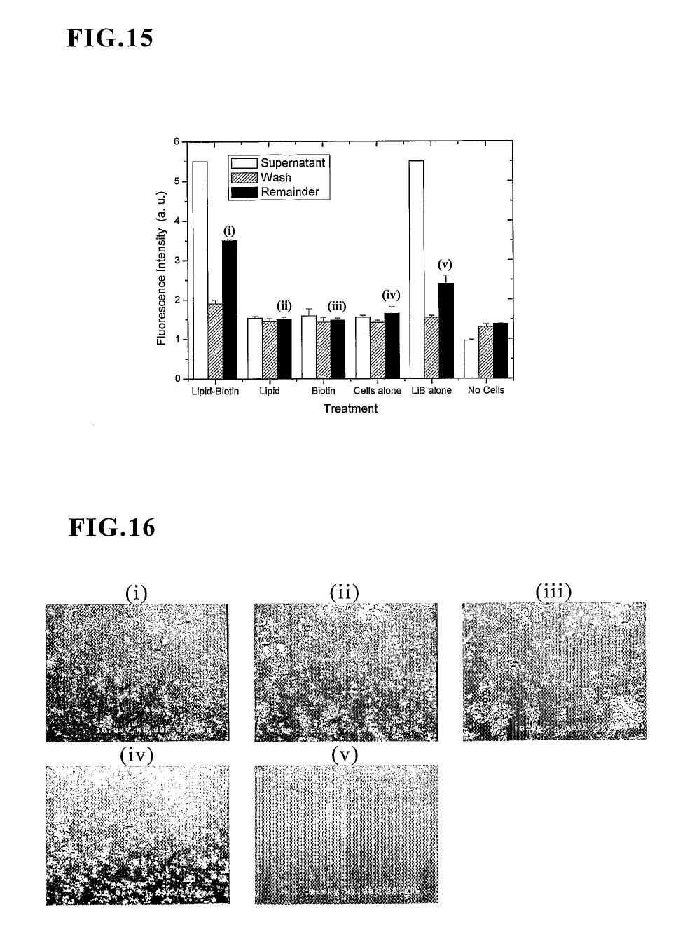

FIG. 15 displays the results of the plate reader experiments. As before, the readings of supernatant and wash can be used as rough guide for the sensitivity range of the experiment. The remainders indicate the fluorescence of Streptavidin attached to the surface-immobilized APL cells via the lipid-biotin probe integrated into their lipid membrane. Again, only in the case of cells bearing the lipid-biotin an increase of fluorescence intensity over the background as indicated by the wash signal can be observed. This shows that the lipid-biotin is required to bind the Streptavidin to the surface-immobilized cells. The controls give evidence that Streptavidin does not adsorb non-specifically on the surface, thereby further corroborating the conclusion that the interaction is specific in nature. When using the lipid-biotin alone, a certain increase in fluorescence intensity on surface can be observed, which can be explained by non-specific adsorption of the lipid-biotin on the surface, which occurs because BSA passivation of the surface works best for proteins and may fail in case of small molecules. For this reason, the cells had been biotinylated prior to surface-immobilization to assure that only membrane-bound biotin is present on the surface.

To assure that the observations are in fact related to surface-adsorbed cells, the bottoms of the cell culture plates were analyzed via SEM after the plate reader experiment. Thereby, the cells were first fixated as detailed in Example 1. Then, the bottom of the plates were cut out, 10 nm of gold deposited, and the surfaces analyzed by SEM. FIG. 16 shows images obtained from the different surfaces. The labels indicate to which of the plate reader results of FIG. 15 the images do correspond. All images exhibit high density of APL cells, except for the last one (FIG. 16(v)), which was the control experiment using no cells but the lipid-biotin only.

As a further validation of the specificity of Streptavidin binding to biotin-labeled APL cells, and thus a further demonstration of the usefulness of APL cells as biofunctional interfaces, the experiment was repeated, this time however including a non-specific interaction test utilizing BSA488. FIG. 17 displays the results of this experiment. In FIG. 17(I), surface-adsorbed APL cells are exposed to SRB as already shown in FIG. 15. As before, only those cells that bear the lipid-labeled biotin in their membranes show an increase in fluorescence over the background, thus indicating bound SRB. In FIG. 17(II), surface-immobilized cells prepared in the same run were exposed to BSA488. This time, however, no increase in fluorescence can be observed, indicating that BSA488 does not non-specifically adsorb to lipid-biotin-labeled APL cells. In a second step, those cells first exposed to SRB were exposed to BSA488 and vice versa. As can be seen from FIG. 17(III), even after binding of SRB the surface-immobilized APL cells remain inert with respect to non-specific binding of BSA488, thereby indicating the fluidity and functionality of the cell membrane of surface-adsorbed APL cells. On the other hand, as shown in FIG. 17(IV), those surface-adsorbed APL cells first exposed to BSA488 are still capable of binding SRB specifically, thus demonstrating the high selectivity of surface-adsorbed APL cell membranes. Note that the two fluorescent dyes used have different excitation wavelength ranges, so that the low fluorescence response for the lipid-biotin-labeled cells in FIG. 17(III), which was obtained with proper settings for BSA488, does not imply that the previously bound Streptavidin left the surface. In fact, a reading of this sample with the plater reader properly set for the detection of SRB gave the same result as found during the first reading prior to BSA488 exposure as shown in FIG. 17(I) (not shown). Therefore, altogether, this experiment demonstrates very nicely the feasibility of utilizing surface-adsorbed APL cells as highly specific biofunctional interfaces.

### Example 5

### Nanopatterning of Acholeplasma Laidlawii

To demonstrate the feasibility of nanopatterning of APL cells, nanopatterns were prepared as follows.

### Experimental:

Polystyrene (PS) beads of different size (500 nm-10 μm) were deposited on clean silicon wafer pieces via drop-coating. The PS beads were used as colloidal mask in a subsequent 50 nm gold evaporation onto the Si wafer pieces. 5 nm of Cr were used as adhesion promoter. After removal from the evaporator, the colloidal mask was removed via 10 min ultrasonication in pure chloroform, leaving Si patches of the diameter of the colloidal particles in the otherwise continuous 50 nm gold film. This inorganic pattern was further cleaned via a UV ozone plasma treatment for 10 min and then immediately exposed to aminopropyltrimethoxysilane (APTMS, CAS-no. 13822-56-5) either via vapor deposition from the liquid phase or via adsorption from 2 mM APTMS/toluene solution. After another 10 min cleaning in pure chloroform, biotin was attached to the APTMS on the Si patches via EDC/NHS coupling. Then, the samples were immersed into a PEG-thiol solution in order to render the gold film cell-resistant. Afterwards, Streptavidin was coupled to the surface-immobilized biotin on the Si patches, thus providing anchor sites for biotin-labeled APL cells. In a future biosensor, such biotin labels inserted into mycoplasma membranes may be further used for integration of ligates into the membrane.

APL-LiB cell suspensions were prepared as described in example 3. Nanopatterns were exposed to APL-LiB by adding them to APL-LiB suspensions in the wells of either a 6-well or 24-well plate (353504, Falcon) and left overnight (0/N) at 37° C. Next day, the chips were washed with PBS from a wash-bottle and then immersed in PBS 4% Glutaraldehyde for 1 hour. After fixation the chips were washed with MQ H₂O from a wash bottle and dried with N₂ from a pressurized cylinder. Surfaces were then coated with 10 nm of gold as described in example 1 and analyzed with SEM.

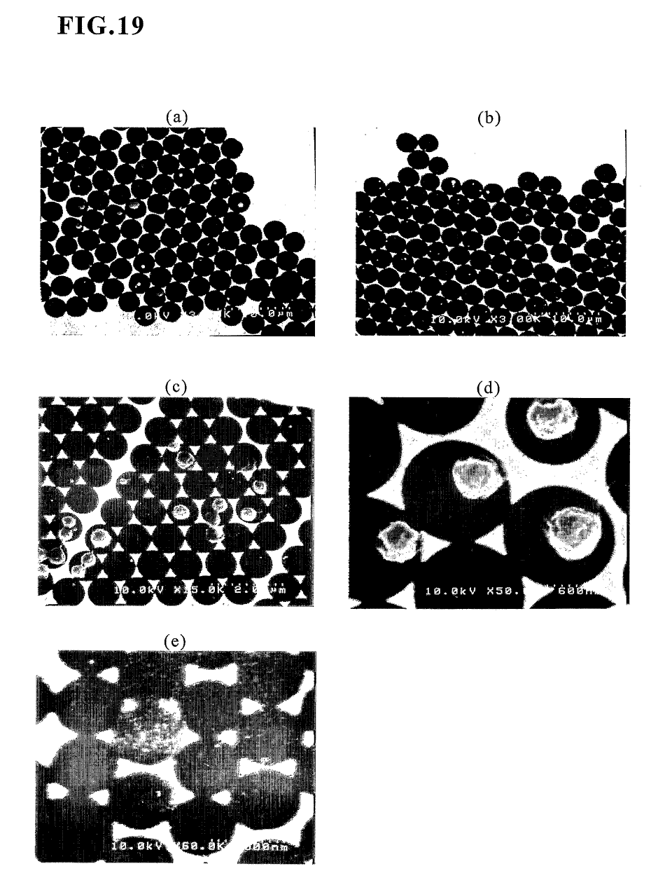

In an alternative scheme (results of which shown in FIG. 19(c) and (d)), the nanopatterns consisting of APMTS-coated Si patches and PEG-coated gold were first exposed to aqueous polyelectrolyte solutions of different charge (first layer poly(styrenesulfonate), second layer Poly(allylamine hydrochloride)) to provide a high density of amino groups on the Si patches. Then, a suspension of EDC/NHS activated APL cells was placed onto the patterns for direct coupling of the cells to the patches.

### Results:

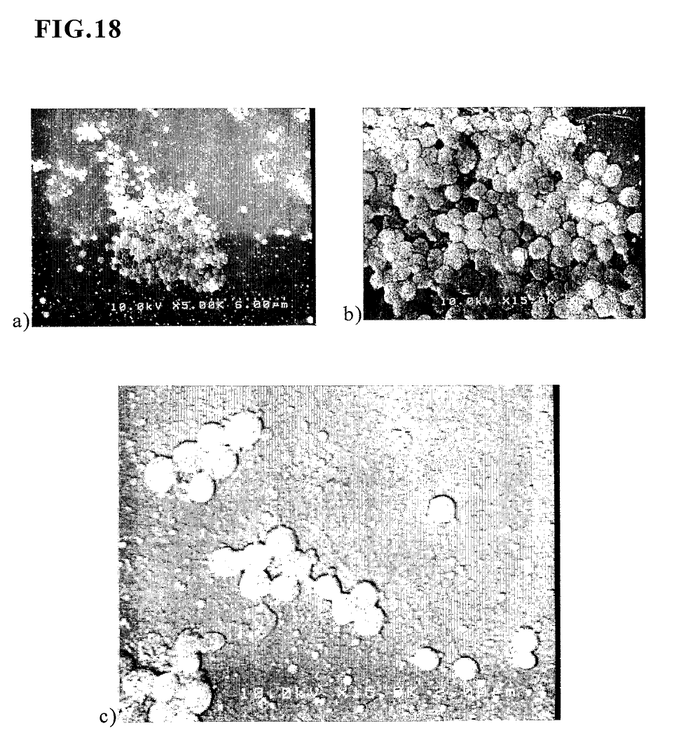

FIG. 19 shows some results of the patterning experiments. While exposure of a non-patterned surface typically causes the immobilization of clusters of cells (cf. FIGS. 12, 16, and 18), nanopatterning yields mainly the adsorption of individual cells. It seems that this trend is somewhat independent of the actual size of the patterned patch, as suggested by the findings shown in FIGS. 19(d) and 19(e). In the latter image, a single cell, which is just of the size of the Si patch (nominal diameter 500 nm) seems to be kept and centered by the gold structures surrounding the patch, while the cells in FIG. 19(d) are not necessarily in contact with the gold structures due to the larger size of the patches of about 1 μm diameter. The density of adsorbed cells can still be improved, for example by increasing the fraction of non-clustered cells in suspension. Also, the adhesion protocol might have to be optimized in the future. Anyway, since no cell adsorption is observed on the PEG-coated gold regions of the surface, the present results demonstrate the selectivity of the surface for APL cell adsorption onto the Si patches, thereby proving the feasibility of the method to generate biofunctional interfaces for biosensing of sub-micron dimension by patterning single APL cells.

### Example 6

### Freezing and Thawing of Acholeplasma Laidlawii

For the practical application of the present embodiment, lifetime and persistence of the properties of the prepared biofunctional interfaces for biosensing is of utmost importance even after long storage times. In the following, we show that APL cultures can be frozen for several days without losing vitality. Accordingly, the cells still provide all membrane functions also necessary for biosensing, such as specificity and fluidity, as such properties are required for higher cell function such as proliferation and growth.

### Experimental:

A 3 day old APL culture in MycoM was taken from the 37° C. water bath and placed in a freezer at −30° C. 3 days later a minimum portion of the frozen culture was taken and used to inoculate MycoM as described in Example 1. 2 days later the culture was treated as described in the first paragraph of Example 2. The cell suspension with an Absorbance at OD260 nm of approx 2.0 was added to a Silicon chip and left for 2 hours at 37° C. before transfer straight into PBS 4% Glutaraldehyde. The chip was then coated with 10 nm of gold and observed by SEM.

### Results:

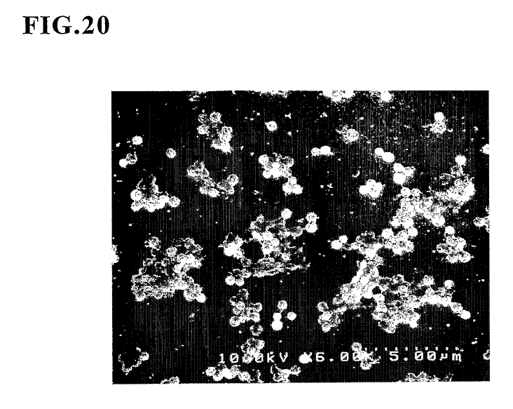

FIG. 20 shows a SEM image of thus obtained culture. The cells have the same appearance as those from cultures that were not frozen before growth (FIG. 12), so that it can be concluded that freezing in MycoM culture medium does not cause any harm to cell function.

Heretofore, the present invention is explained with reference to the embodiments. However, various changes or improvements can be applied to the embodiments.

1. A biosensor chip for sensing a target molecule, comprising:
a substrate having a surface with a sensing area;
and adhesive material for immobilizing a mollicute having a cell membrane on the sensing area.

2. The biosensor chip according to claim 1, further comprising:
cell-resistant material for preventing the mollicute from being immobilized on those parts of the surface of the substrate that do not belong to the sensing area.

3. The biosensor chip according to claim 1, wherein
the adhesive material comprises a first adhesive material for immobilizing a body of the mollicute on the sensing area and a second adhesive material for immobilizing a tip of the mollicute on the surface of the substrate.

4. The biosensor chip according to claim 1, comprising:
the mollicute immobilized with the adhesive material on the sensing area.

5. The biosensor chip according to claim 4, comprising:
a biomolecule capable of specific recognition embedded into the cell membrane of the mollicute.

6. The biosensor chip according to claim 1, wherein
a plurality of sensing areas are embedded into the substrate; and
the adhesive material is disposed on only a part of the sensing areas.

7. The biosensor chip according to claim 1, wherein
the substrate comprises a particle having a surface to define an optical cavity to confine light in the surface of the particle by resonant recirculation; and
a part of the surface of the particle is exposed to the outside of the surface of the substrate as to constitute the sensing area by the part of the surface of the particle.

8. A biosensor for sensing a target molecule, comprising:
the biosensor chip according to claim 5;
a transducer for detecting changes in mass or refractive index on the sensing area; and
a flow cell providing the biosensor chip with analyte.

9. A method for producing a biosensor chip for sensing a target molecule, comprising:
preparing a substrate having a surface with a sensing area; and
disposing adhesive material on the sensing area for immobilizing a mollicute having a cell membrane on the sensing area.

10. The method for producing the biosensor chip according to claim 9, further comprising:
disposing cell-resistant material on those parts of the surface of the substrate that do not belong to the sensing area for preventing the mollicute from being immobilized on parts of the surface of the substrate.

11. The method for producing the biosensor chip according to claim 9, wherein
the adhesive material comprises a first adhesive material for immobilizing a body of the mollicute on the sensing area and a second adhesive material for immobilizing a tip of the mollicute on the surface of the substrate.

12. The method for producing the biosensor chip according to claim 9, comprising:
immobilizing the mollicute with the adhesive material on the sensing area.

13. The method for producing the biosensor chip according to claim 12, comprising:
embedding a biomolecule capable of specific recognition into the cell membrane of the mollicute by attaching the biomolecule to a lipid molecule so that the lipid molecule can be assembled into the cell membrane of the mollicute.

14. The method for producing the biosensor chip according to claim 12, comprising:
embedding a biomolecule capable of specific recognition into the cell membrane of the mollicute by modifying a inherent DNA sequence of the mollicute by at least one sequence required for the expression of the biomolecule in a cell membrane or by transforming a plasmid or bacteriophage into the mollicute so that the mollicute can express the biomolecule in a cell membrane of the mollicute.
